(19) United States
(12) Patent Application Publication
(10) Pub. No.: US 2007/0238967 A1
(43) Pub. Date: Oct. 11, 2007

(54) Overall medical system

(75) Inventors: Stefan Sattler, Forchheim (DE); Reiner Staab, Baiersdorf (DE)

Correspondence Address: SIEMENS CORPORATION;INTELLECTUAL PROPERTY DEPARTMENT, 170 WOOD AVENUE SOUTH, ISELIN, NJ, 08830, US

(73) Assignee: SIEMENS AKTIENGESELLSCHAFT

(21) Appl. No.: 11/732,944

(22) Filed: Apr. 5, 2007

(30) Foreign Application Priority Data
Apr. 6, 2006 (DE) 1020060016331.1

(51) Int. Cl. A61B 5/05

(52) U.S. Cl. 600/407; 600/420

(57) ABSTRACT

To ensure a flexible and patient-friendly examination by means of an overall medical system having at least one medical examination or treatment facility, having a control facility and an assigned medical injection system, provision is made for the injection system to be able to be coupled to the overall system by means of a wireless bidirectional communication and/or control link.

### CROSS REFERENCE TO RELATED APPLICATIONS

This application claims priority of German application No. 10 2006 0016 331.1 filed Apr. 6, 2006, which is incorporated by reference herein in its entirety.

### FIELD OF THE INVENTION

The invention relates to an overall medical system having a control facility, at least one medical examination or treatment facility and a medical injection system.

### BACKGROUND OF THE INVENTION

In the case of radiological examinations, contrast media are frequently administered. This so-called bolus injection allows a stronger contrast with the recording of vessels or tissues using a radiological diagnosis device. In addition to improving the contrast, details relating for instance to disruptions in the blood-tissue barriers can be obtained with a temporally well-defined bolus injection from the temporal sequence in which the contrast medium spreads in the tissue. The temporal coordination, in other words the timing, of the bolus injection and the start of the measurements with the radiological diagnosis device is fundamental to this technology in terms of reproducibility and high quality.

The bolus injection can either be carried out manually by the doctor using an injection or can be given by means of a mechanical medical injection system, which is controlled for instance by way of a control unit. In order to avoid stressing the patient for longer than necessary and in order to achieve a utilizable examination result, the most precise and reliable synchronization possible is required between the injection administration, the introduction of the x-ray beam and the start of the x-ray image recording. For this reason, the medical injection system is generally connected to the control facility of the examination device for communication and data exchange purposes by means of a cable. Such a device is known for instance from document DE 103 02 636 A1. Data transmission by cable is disadvantageous for practical accident avoidance and for hygiene reasons.

### SUMMARY OF THE INVENTION

The object underlying the present invention is to enable a flexible, secure and patient-friendly examination with an overall medical system having a medical injection system comprising simple means.

This object is achieved in accordance with the invention by an overall medical system having a control facility, at least one medical examination or treatment facility and having a medical injection system according to the claims. Advantageous embodiments of the invention are the subject matter of the subclaims in each instance.

With the overall system according to the invention, an impractical, accident-prone and unhygienic cable can be dispensed with in the event of a collectively ensured synchronization of the examination processes by means of the bidirectional communication and/or control link by way of wireless data transmission and, in addition, the flexibility of the examination can be increased by facilitating a positioning and a transportation of the medical injection device without the interfering cable.

According to one embodiment of the invention, the overall system comprises a wireless bidirectional communication and/or control link between the injection system and the control facility. A particularly effective synchronization of the temporal coordination of the bolus injection and the start of the measurements of the examination or treatment facility is herewith ensured.

Advantageous cable-free communication and/or control links are formed above all by radio links or infrared links. The said cable-free links can be integrated into the overall system in a flexible and uncomplicated fashion.

According to a further embodiment of the invention, the communication and/or control link is designed in a direction-selective or frequency-selective manner, such that the transmission reliability is increased and a clear assignment of the medical injection system to the overall system is simplified.

The medical injection system expediently comprises a cable-independent voltage supply, in particular in the form of an accumulator integrated into the medical injection system. The combination of a wireless communication/control and a wireless voltage supply enables a perfect galvanic and mechanical decoupling of the medical injection system from the overall system.

The communication and/or control link between the injection system and the control facility advantageously comprises a complex data protocol based on the CANopen (Controller Area Network open) standard, for improved reliability in terms of wireless communication and/or control. A bidirectional transmission of trigger signals also serves the same purpose.

According to a further embodiment of the invention, the injection system comprises an RFID transponder (Radio Frequency Identification) and the control facility comprises an RFID transmit/receive unit. This enables a simple and unambiguous identification of the medical injection system in respect for example of the injector or of the contrast medium to be guaranteed. In this way, the availability of the medical injection system can also be checked, and the assignment facilitated.

The invention is particularly advantageous for an overall system, with which the examination or treatment facility is formed by a computed tomography system or by a magnetic resonance system or by a PET system (Positron Emission Tomography) or by a SPECT system (Single Photon Emission Computed Tomography) or by an x-ray device.

### BRIEF DESCRIPTION OF THE DRAWINGS

The invention and further advantageous embodiments according to the features of the subclaims are described in more detail below with reference to schematically illustrated exemplary embodiments in the drawing, without herewith restricting the invention to these exemplary embodiments, in which:

FIG. 1 shows an overall medical system according to the invention having a medical injection system and an x-ray diagnosis device

FIG. 2 shows a detailed view of the medical injection system for an overall medical system according to the invention.

### DETAILED DESCRIPTION OF THE INVENTION

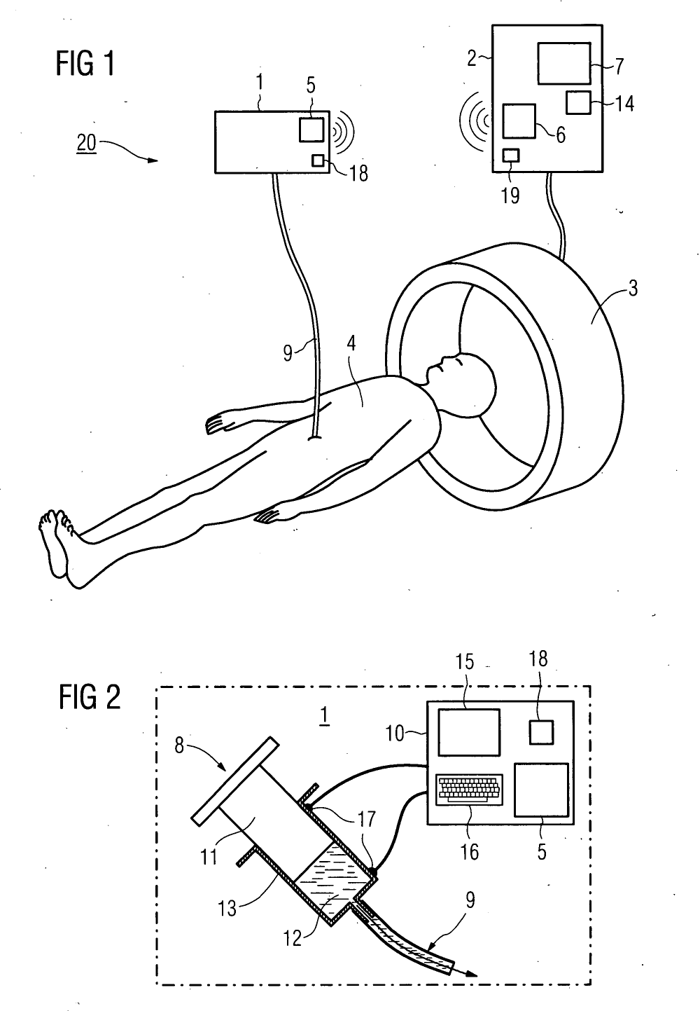

FIG. 1 shows an overall medical system 20 according to the invention, which comprises a medical examination or treatment facility 3, a control facility 2 and an assignable medical injection system 1 as essential components. The control facility 2 serves to control and monitor the overall system 20 and to this end features a control computer 7 having a display and an image system 14 for image processing purposes. The control facility 2 is connected to the medical examination or treatment facility 3 by means of a cable, but can however be connected in a cable-free manner or integrated into the medical examination or treatment facility 3.

In accordance with the invention, the control facility 2 can be coupled to the medical injection system 1 by means of a wireless bidirectional communication and/or control link. To this end, the medical injection system 1 has a first transmit and receive means 5 and the control facility has a second transmit and receive means 6. Depending on the wireless communication method, the transmit and receive means 5; 6 can be configured for instance as a radio transmitter/receiver or as an infrared interface.

According to one embodiment of the invention, the communication and/or control between the injection system 1 and the control facility 2 exhibits a complex data protocol based on the CANOpen (Controller Area Network Open) standard for injectors. The standard “CiA DSP 425 (CANOpen in Automation Draft Standard Proposal) Part 2: Injector” is known here for instance. Provision can also be made for the communication and/or control between the injection system 1 and the control facility 2 to comprise a bidirectional transmission of trigger signals. Further possibilities are transmissions by WLAN or Bluetooth.

For fault-free assignment and coupling to the overall system 20, the medical injection system 1 can additionally comprise an RFID transponder (Radio Frequency Identification) 18. Information relating to the injector is stored on the RFID transponder 18. The control facility 2 also additionally features an RFID transmit/receive unit 19. With the assignment of the medical injection system 1 to the overall system 20, the control facility 2 checks by means of the RFID transmit/receive unit 19 whether the suitable injection system 1 is also available in order to avoid potential serious errors as a result of incorrect contrast medium administration.

For examination purposes, a contrast medium 12 is administered to the patient 4 via a supply line 9 and an infusion needle (not shown) by means of the medical injection system. The trigger command of the injection and/or other relevant parameters of the examination are transmitted in a cable-free manner to the medical injection system 1 by means of the control facility 2. The medical injection system 1 likewise transmits its data, such as for instance trigger time or injection quantity, to the control facility 2 in a cable-free manner. The control facility 2 subsequently controls the synchronization with the examination or treatment facility 3 as a function for instance of the injection time.

By way of example, FIG. 2 shows a detailed medical injection system 1. The medical injection system 1 comprises an injector 8 and a control device 10. The control device 10 in turn features the first transmit and receive means 5, a display 15 and an input unit, in particular a 16. The injector 8 includes a piston 11, a cylinder 13 and a contrast medium 12. The contrast medium 12 is injected intravenously into the patient 4 via the supply line 9 by inserting the piston 11 into the cylinder 14. The control unit 10 can check the fill level of the contrast medium 12 by way of a sensor 17 for instance.

On example of a medical injection system 1 is an injection pump, in particular the so-called perfusor. Medical injection systems 1 are also known which do not feature any individual control device 10 but are instead controlled and monitored by means of the CANOpen standard by way of the overall medical system 20. Furthermore, medical injectors 1 are also known, which can inject several substances such as saline solution for instance on the one hand and a contrast medium 12 on the other hand (dual head injector).

The invention can be summarized as follows: to ensure a flexible and patient-friendly examination by means of an overall medical system 20 having at least one medical examination or treatment facility, a control facility 2 and having an assigned medical injection system 1, provision is made for the injection system 1 to be able to be coupled to the overall system 20 by means of a wireless bidirectional communication and/or control link.

1.-10. (canceled)

11. A medical system used in a medical examination for a patient, comprising:
a medical examination device that performs the medical examination for the patient; and
a medical injection system that injects a contrast medium to the patient and is coupled to the medical system by a wireless bidirectional link.

12. The medical system as claimed in claim 11, wherein the wireless bidirectional link connects the injection system to a control unit of the medical system.

13. The medical system as claimed in claim 12,
wherein a signal is transmitted by the wireless bidirectional link between the injection system and the control unit, and
wherein the signal is selected form the group consisting of: a trigger command of the injection, an injection time, and an injection quantity.

14. The medical system as claimed in claim 12, wherein the injection system comprises an RFID transponder and the control unit comprises an RFID transmit/receive unit.

15. The medical system as claimed in claim 11, wherein the wireless bidirectional link comprises a radio connection or an infrared connection.

16. The medical system as claimed in claim 11, wherein the wireless bidirectional link is direction selective or frequency selective.

17. The medical system as claimed in claim 11, wherein the wireless bidirectional link comprises a data protocol based on a CANopen standard.

18. The medical system as claimed in claim 11,
wherein the injection system comprises a cable independent voltage supply, and
wherein the cable independent voltage supply is an accumulator.

19. The medical system as claimed in claim 11, wherein the medical examination device is selected from the group consisting of: a computed tomography device, a magnetic resonance device, a positron emission tomography device, an individual photon emission computed tomography device, and an x-ray device.

20. The medical system as claimed in claim 11, wherein the wireless bidirectional link is a communication link or a control link.

21. A method for operating a medical system used in a medical examination for a patient, comprising:
injecting a contrast medium into the patient by an injecting system;
connecting the injecting system to the medical system via a wireless bidirectional link; and
synchronizing the injection with the medical examination via the wireless bidirectional link.

22. The method as claimed in claim 21, wherein the injection system is connected to a control unit of the medical system via the wireless bidirectional link.

23. The method as claimed in claim 22,
wherein a signal is transmitted by the wireless bidirectional link between the injection system and the control unit, and
wherein the signal is selected form the group consisting of: a trigger command of the injection, an injection time, and an injection quantity.

24. The method as claimed in claim 22, wherein the injection system comprises an RFID transponder and the control unit comprises an RFID transmit/receive unit.

25. The method as claimed in claim 21, wherein the wireless bidirectional link comprises a radio connection or an infrared connection.

26. The method as claimed in claim 21, wherein the wireless bidirectional link is direction selective or frequency selective.

27. The method as claimed in claim 21, wherein the wireless bidirectional link comprises a data protocol based on a CANopen standard.

28. The method as claimed in claim 21,
wherein the injection system comprises a cable independent voltage supply, and
wherein the cable independent voltage supply is an accumulator.

29. The method as claimed in claim 21, wherein the medical examination device is selected from the group consisting of: a computed tomography device, a magnetic resonance device, a positron emission tomography device, an individual photon emission computed tomography device, and an x-ray device.

30. The method as claimed in claim 21, wherein the wireless bidirectional link is a communication link or a control link.
